(19) United States
(12) Patent Application Publication
(10) Pub. No.: US 2007/0148721 A1
(43) Pub. Date: Jun. 28, 2007

(54) Compositions, methods, systems, and kits for affinity purification

(75) Inventors: Gordon R. Nicol, Middletown, DE (US); Barry E. Boyes, Wilmington, DE (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(73) Assignee: Agilent Technologies, Inc., Palo Alto, CA (US)

(21) Appl. No.: 11/319,885

(22) Filed: Dec. 28, 2005

(51) Int. Cl. C12Q 1/37

(52) U.S. Cl. 435/23; 530/416

(57) ABSTRACT

The invention provides a method of separating proteins and peptides of a sample comprising contacting the sample with a Pd coordination compound. The Pd coordination compound binds to sulfur and/or nitrogen groups and thus is useful for purifying biomolecules comprising cysteine, methionine, histidine amino acids or derivatized amino acids/residues comprising sulfur or nitrogen groups which bind to coordination sites on the Pd coordination compounds. The invention also relates to methods, systems and kits for using the Pd coordination compound.

### BACKGROUND

Proteins regulate biological processes, provide the structural components of cells, and control metabolic functions. Protein activity is not always directly correlated with the expression level of a corresponding mRNA transcript in a cell, but is impacted by post-translational modifications, such as protein phosphorylation, processing events (e.g., cleavage) and the association of proteins with other biomolecules. The large-scale analysis of all of the proteins expressed by a genome, including modified and unmodified, processed and unprocessed forms, has been termed “proteome analysis.”

Quantitative proteomics or proteome profiling is the systematic analysis of all proteins expressed by a cell or tissue with respect to their quantity and identity and form. By examining proteome states in different cells, for example, at different developmental stages, under pathological conditions, after exposure to different agents (drugs, toxic chemicals, environmental conditions, and the like), changes in protein expression may be related to changes in cell states (e.g., a progression from a normal state to a pathological state).

Because proteome profiling requires the analysis of thousands of proteins, high throughput techniques for obtaining information regarding protein identity and quantity in a sample are required. Traditionally, samples have been resolved by two-dimensional polyacrylamide gel electrophoresis (2D-PAGE), and the relative concentrations of proteins of interest determined by generic protein staining and densitometry or fluorescence intensity, which can be limited by the dynamic range of the 2D gel/staining and scanning. In addition, there is a high probability that more than one protein is present in any particular spot on the gel. Further, 2D-PAGE is difficult to automate and does not lend itself readily to parallel evaluation of large numbers of samples. Additionally, lower abundant proteins (such as important regulatory proteins) are often outside of the range of detection of 2D-PAGE.

Mass spectrometry permits a high throughput approach to proteome analysis. Mass spectrometry may be used to evaluate whole proteins, e.g., by surface-enhanced laser desorption ionization-time of flight mass spectrometry (SELDI-TOF); however, MS analysis of whole proteins does not directly provide sequence-based identification.

In tandem mass spectrometry techniques (MS/MS), protein samples are digested with chemicals or enzymes (e.g., trypsin), and peptides obtained are further fragmented in a mass spectrometer. Referential analysis between MS fragmentation patterns and the in silico generation of theoretical peptide fragments from reverse translation/transcription of genomic data may be used to identify and quantify proteins, since peptides may provide signatures for proteins from which they are derived.

Techniques for characterizing and comparing proteomes that rely on mass spectroscopy include ICAT technology (see, e.g., U.S. Pat. No. 6,670,194 by Aebersold, et al.), which is based on the use of isotope-coded affinity tags (ICATs) in combination with MS/MS. The ICAT method is based on the modification of cysteine-containing proteins by an iodacetate derivative carrying a biotin label. After enzymatically cleaving modified proteins into peptides, cysteine-modified, biotin-labeled peptides are purified using avidin-coated beads. This affinity purification step reduces the complexity of the original peptide mixture, making the sample more amenable to mass spectrometry. Comparative quantification may be performed by differentially labeling two samples being compared, e.g., with light and heavy isotope labels, such that peptides that are the same chemically (i.e., have the same amino acid sequence) will have different masses, distinguishable by mass spectrometric analysis.

The avidin-biotin interaction is relatively strong and allows the sample to be washed so that the peptides that do not bind to the avidin resin will be removed leaving only peptides that contain the cysteine residue that has been derivatized with this reagent. The avidin-biotin interaction is not completely specific and peptides that do not contain the biotin moiety will also bind to avidin. In addition, due to the strength of the avidin-biotin interaction, disassociation of avidin-biotin complexes is difficult. Furthermore, peptides are often found that do not contain a cysteine, which are selected due to the non-specific interactions between such peptides and avidin or the solid phase to which it is attached.

The ICAT technology limits analysis of proteomes to those peptides that contain a cysteine residue. Cysteines are found in 85% of the proteins in many mammalian species, exhibiting an overall occurrence in the human proteome of about 1.7%. That is, for every 1000 amino acids in the human proteome there are only 17 cysteines. This makes both identification and quantification of proteins difficult.

### SUMMARY

In one embodiment, the invention provides methods, systems, compositions and kits for affinity purification of proteins and/or peptides. These methods, compositions and kits may be used in proteome analysis, for example, in conjunction with mass spectroscopy techniques.

In one embodiment, the invention provides a composition comprising a palladium (Pd) coordination compound. In certain embodiments the Pd coordination compound is stably associated with a substrate (“Pd-substrate”). The Pd-substrate composition provides an affinity matrix for selectively binding to proteins or peptide fragments thereof which comprise sulfur or an —NH₂ group, an imidazole, or nitrogen group capable of donating electron groups to a Pd compound coordination compound, such as proteins or peptides comprising methionines, histidines, or reduced cysteines.

In another embodiment, two different samples are affinity purified to obtain two populations of proteins enriched in sulfur and nitrogen groups, e.g., to enrich for proteins comprising cysteine, methionine, and histidine. The two different samples may be differentially labeled, e.g., both populations may comprise different types of labels, or one population may comprise labeled proteins, while the other population comprises unlabeled proteins. In one aspect, a label comprises a molecule that alters the mass of proteins or peptide fragments thereof. In another aspect, the label comprises an isotopic label. Proteins may be labeled before, after, or during binding to the affinity column. In one aspect, the label is one that permits discrimination between two identical but differentially labeled peptide fragments of a protein. For example, differentially labeled peptides may generate distinct mass spectra peaks. In certain embodiments, differential labeling is carried out across multiple different samples to generate substantially chemically identical peptides that are distinguishable by mass. The populations may be mixed together to determine ratios of peptides labeled with first and second labels, or ratios of unlabeled and labeled peptides. In certain aspects, mass altering labels are selected which are used to differentiate between modified and unmodified forms of proteins. In other aspects, protein modifications alter the mass of peptides. Internal standards representing modified or unmodified proteins, or fragments thereof, may be spiked into protein samples to provide a means to calibrate amounts of modified proteins.

In one aspect, two or more protein samples are compared, for example, a protein sample or source from which the sample is obtained which has been exposed to an agent (a drug, carcinogen, potential toxin, potential carcinogen, teratogen, hormone) or condition (e.g. an environment, treatment regimen, temperature, etc.). The protein samples can also be derived from normal cells in different states of differentiation, or derived from normal and diseased cells, or diseased and drug-treated cells or some combination thereof. Samples may be compared by differentially labeling the samples, for example, with first and second mass-altering labels (e.g., heavy and light isotope pairs) and determining the ratio of an amount of peptide having a first label to an amount of peptide having a second label. The identification of peptides whose expression (amount and/or form) is different between samples may be used to identify peptides important in changes in cell state and/or in responses to disease, agents, environmental conditions and the like. In some embodiments, proteome samples may be screened with compounds (e.g., libraries of biomolecules), to identify those compounds that are able to produce a desired cell state.

In another aspect, two or more protein samples are compared a first sample comprises a first developmental stage and the second sample comprises a second developmental stage. Developmental stages include, for example, embryonic stages: zygote, 2-cell, 4-cell, etc.

In a further embodiment, the invention provides a system comprising a Pd coordination compound stably associated with a substrate for binding proteins and/or peptides, and an analysis system for determining at least one characteristic of a protein or peptide separated and/or recovered using the Pd coordination compound (e.g., such as mass, sequence, quantity, etc.). The system may comprise modular components which may be connectable to each other via interfacing modules. In one aspect, the system further comprises a separation device for separating proteins or peptides separated using the Pd coordination compound. In another aspect, the system comprises an analysis device for determining at least one characteristic of a protein or peptide separated using the Pd-substrate column. In one aspect, the analysis device comprises a mass spectrometer. The separation device may be interfaced with the detection analysis device by, for example, an electrospray device. Either or both the Pd-substrate and separation device may be contained within a microfluidic device and may be connected by channels in a microfluidic substrate. In another aspect, the system further comprises a processor for receiving data from the analysis device. In certain aspects, the processor sends instructions to one or more system components for controlling a system function. In one aspect, the processor receives data relating to a system function. In another aspect, the processor provides or alters instructions to system components in response to data received from the system. In a further aspect, the processor accesses a memory which may be remote from the processor and/or other system components.

In a further embodiment, the invention provides kits for facilitating methods according to embodiments of the invention. In one aspect, a kit comprises a Pd-substrate composition and one or more reagents, such as a cell lysis buffer, an elution solution for removing a protein or peptide bound to a Pd-substrate, or buffers compatible with MS systems. A kit also provides instructions on the use of these composite elements to provide application to analysis and interpretation of the measurements that can be obtained on a proteome sample or samples.

### BRIEF DESCRIPTION OF THE FIGURES

The objects and features of the invention can be better understood with reference to the following detailed description and accompanying drawings.

FIG. 1 illustrates a reaction scheme for the binding of sulfur-containing peptides with a Pd compound (Compound I) according to one aspect of the invention.

FIG. 2 illustrates a reaction scheme for the selection of sulfur containing peptides on to a Pd complex anchored to a substrate (SP) (n=any integer≧0).

FIG. 3A shows a Pd coordination compound stably associated with a substrate via a flexible linker (L). FIG. 3B shows reactive complexes which may than be attached to a solid support.

FIG. 4 illustrates a scheme for the reaction of cysteine residues with a methanethiosulfonate (MTS) reagent, methyl methanethiosulfonate, in order to block binding of cysteine to the Pd resin.

FIG. 5A illustrates a scheme for the reaction of cysteine residues with iodoacetic acid in order to prevent oxidation of cysteine residues via disulfur linkage, thus preserving the ability of cysteine to bind to the Pd resin. FIG. 5B illustrates a scheme for the reaction of cysteine residues with vinyl pyridine in order to prevent oxidation of cysteine residues via disulfur linkage, thus preserving the ability of cysteine to bind to the Pd resin.

FIG. 6 is a schematic demonstrating the steps for how proteins containing methionine, cysteine, or histidine can be prepared for analysis using the methods of the invention.

FIG. 7 is a schematic demonstrating the steps for how proteins containing methionine or histidine can be prepared for analysis using the methods of the invention.

### DETAILED DESCRIPTION OF EMBODIMENTS OF THE INVENTION

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. Methods recited herein may be carried out in any order that is logically possible, in addition to a particular order disclosed.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The practice of the present invention will employ, unless otherwise indicated, conventional techniques of chemistry, biochemistry, molecular biology, and medicine, including diagnostics, which are within the skill of the art. Such techniques are explained fully in the literature.

The following definitions are provided for specific terms that are used in the following written description.

It must be noted that as used herein and in the appended claims, the singular forms “a”, “and”, and “the” include plural referents unless the context clearly dictates otherwise. Thus, for example, reference to “a protein” includes a plurality of such proteins and reference to “protein” includes reference to one or more proteins and equivalents thereof known to those skilled in the art, and so forth.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

It will also be appreciated that throughout the present application, that words such as “cover”, “base” “front”, “back”, “top”, “upper”, and “lower” are used in a relative sense only.

A “set” or “sub-set” of any item (such as a set of proteins or peptides) may contain only one of the item, or only two, or three, or any multiple number of the items.

As used herein, a “peptide mixture” is typically a complex mixture of peptides obtained as a result of the cleavage of a sample comprising proteins.

As used herein, a “sample of proteins” is typically any complex mixture of proteins and/or their modified and/or processed forms, which may be obtained from sources, including, without limitation: a cell sample (e.g., lysate, suspension, collection of adherent cells on a culture plate, a scraping, a fragment or slice of tissue, a tumor, biopsy sample, an archival cell or tissue sample, laser-capture dissected cells, etc), an organism (e.g., a microorganism such as a bacteria or yeast), a subcellular fraction (e.g., comprising organelles such as nuclei or mitochondria, large protein complexes such as ribosomes or golgi, and the like), an egg, sperm, embryo, a biological fluid, viruses, and the like.

The term “peptide” as used herein refers to an entity comprising at least one peptide bond, and can comprise D and/or L amino acids. A “ligand” is a peptide consisting essentially of about 2 to about 20 amino acids (i.e., about 2, 3, 4, 5, 6, 7, 8, 9, 10, 11, 12, 13, 14, 15, 16, 17, 18, 19, 20 amino acids).

“Protein”, as used herein, means any protein, including, but not limited to peptides, enzymes, glycoproteins, hormones, receptors, antigens, antibodies, growth factors, etc., without limitation. Proteins include those comprised of greater than about 20 amino acids. The terms “polypeptide” and “protein” are generally used interchangeably herein. The term “proteins or peptides or a sample” refers to a sample comprising proteins only, peptides only, or a mixture of proteins and peptides.

As used herein, “a peptide fragmentation signature” refers to the distribution of mass-to-charge ratios of fragmented peptide ions obtained from fragmenting a peptide, for example, by collision induced disassociation, ECD, LID, PSD, IRNPD, SID, and other fragmentation methods. A peptide fragmentation signature which is “diagnostic” or a “diagnostic signature” of a target protein or target polypeptide is one which is reproducibly observed when a peptide digestion product of a target protein/polypeptide identical in sequence to the peptide portion of a peptide internal standard, is fragmented or which differs only from the fragmentation pattern of the peptide internal standard by the mass of the mass-altering label.

As used herein, a peptide is said to be “isolated” or “substantially purified” when it is substantially free of cellular material or free of chemical precursors or other chemicals. The peptides of the present invention can be purified to homogeneity or other degrees of purity. The level of purification will be based on the intended use. The critical feature is that the preparation allows for the desired function of the peptide, even if in the presence of considerable amounts of other components.

In certain embodiments the purified peptide contains no more than about 50% impurities. In another embodiment no more than about 10% and in yet another embodiment no more than about 1% impurities (all % are weight percentages).

As used herein, a “a biological fluid” includes, but is not limited to, blood, plasma, serum, sputum, urine, tears, saliva, sputum, cerebrospinal fluid, lavages, leukapheresis samples, milk, ductal fluid, perspiration, lymph, semen, umbilical cord fluid, and amniotic fluid, as well as fluid obtained by culturing cells, such as fermentation broth and cell culture medium.

As used herein, “a sample of complex proteins” may contain greater than about 100, about 500, about 1,000, about 5,000, about 10,000, about 20,000, about 30,000, about 100,000 or more different proteins. Such samples may be derived from a natural biological source (e.g., cells, tissue, bodily fluid, soil or water sample, and the like) or may be artificially generated (e.g., by combining one or more samples of natural and/or synthetic or recombinant sources of proteins).

As used herein, “expression” refers to a level, form, or localization of product. For example, “expression of a protein” refers to one or more of the level, form (e.g., presence, absence or quantity of modifications, or cleavage or other processed products), or localization of the protein.

As used herein, a “difference in expression” or “differential expression” refers to an increase or decrease in expression. A difference may be an increase or a decrease in a quantitative measure (e.g., amount of a protein or modified or processed form thereof) or a change in a qualitative measure (e.g., a change in the localization of a protein). Where a difference is observed in a quantitative measure, the difference according to the invention will be at least about 10% greater or less than the level in a normal standard sample. Where a difference is an increase, the increase may be as much as about 20%, 30%, 50%, 70%, 90%, 100% (2-fold) or more, up to and including about 5-fold, 10-fold, 20-fold, 50-fold or more. Where a difference is a decrease, the decrease may be as much as about 20%, 30%, 50%, 70%, 90%, 95%, 98%, 99% or even up to and including 100% (no specific protein or RNA present). It should be noted that even qualitative differences may be represented in quantitative terms if desired. For example, a change in the intracellular localization of a protein may be represented as a change in the percentage of cells showing the original localization.

As used herein, “a diagnostic trait” is an identifying characteristic, or set of characteristics, which in totality, are diagnostic. The term “trait” encompasses both biological characteristics and experiences (e.g., exposure to a drug, occupation, place of residence). In one aspect, a trait is a marker for a particular cell type, such as a transformed, immortalized, pre-cancerous, or cancerous cell, or a state (e.g., a disease) and detection of the trait provides a reliable indicia that the sample comprises that cell type or state. Screening for an agent affecting a trait thus refers to identifying an agent which can cause a detectable change or response in that trait which is statistically significant within 95% confidence levels.

As used herein, the term “cancer” refers to a malignant disease caused or characterized by the proliferation of cells that have lost susceptibility to normal growth control. “Malignant disease” refers to a disease caused by cells that have gained the ability to invade either the cells of origin or to travel to sites removed from the cells of origin.

As used herein, a “cancer-specific marker” is a biomolecule which is expressed preferentially on cancer cells and is not expressed or is expressed to a small degree in non-cancer cells of an adult individual. As used herein, “a small degree” means that the difference in expression of the marker in cancer cells and non-cancer cells is large enough to be detected as a statistically significant difference when using routine statistical methods to within 95% confidence levels.

As used herein “a correlation” refers to a statistically significant relationship determined using routine statistical methods known in the art. For example, in one aspect, statistical significance is determined using a Student's unpaired t-test, considering differences as statistically significant at p<0.05.

As used herein, a “diagnostic probe” is a probe which upon binding to a tissue and/or cell sample provides an indication of the presence or absence of a particular trait. In one aspect, a probe is considered diagnostic if it binds to a diseased tissue and/or cell (“disease samples”) in at least about 80% of samples tested comprising diseased tissue/cells and binds to less than 10% of non-diseased tissue/cells in samples (“non-disease” samples). Preferably, the probe binds to at least about 90% or at least about 95% of disease samples and binds to less than about 5% or 1% of non-disease samples.

The term “isotopic forms” refers to multiple versions of a derivatizing agent which are identical structurally but differ in isotopic content.

The term “proteome” refer to the protein constituents expressed by a genome, typically represented at a given point in time. A “sub-proteome” is a portion or subset of the proteome, for example, the proteins involved in a selected metabolic pathway, or a set of proteins having a common enzymatic activity.

A “remote location,” refers to location other than the location at which the affinity purification and/or mass spectroscopy occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating information” refers to transmitting the data representing that information as signals (e.g., electrical, optical, radio, magnetic, etc) over a suitable communication channel (e.g., a private or public network).

As used herein, a component of a system which is “in communication with” or “communicates with” another component of a system receives input from that component and/or provides an output to that component to implement a system function. A component which is “in communication with” or which “communicates with” another component may be, but is not necessarily, physically connected to the other component. For example, the component may communicate information to the other component and/or receive information from the other component. “Input” or “Output” may be in the form of electrical signals, light, data (e.g., spectral data), materials, or may be in the form of an action taken by the system or component of the system. The term “in communication with” also encompasses a physical connection that may be direct or indirect between one system and another or one component of a system and another.

“Forwarding” an item refers to any means of getting that item from one location to the next, whether by physically transporting that item or otherwise (where that is possible) and includes, at least in the case of data, physically transporting a medium carrying the data or communicating the data.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture. In certain instances a computer-based system may include one or more wireless devices.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

As used herein, a “database” is a collection of information or facts organized according to a data model which determines whether the data is ordered using linked files, hierarchically, according to relational tables, or according to some other model determined by the system operator.

As used herein, an “information management system” refers to a program, or series of programs, which can search a database and determine relationships between data identified as a result of such a search.

As used herein, an “interface on the display of a user device” or “user interface” or “graphical user interface” is a display (comprising text and/or graphical information) displayed by the screen or monitor of a user device connectable to the network which enables a user to interact with a system processor and/or system memory (e.g., including a data base and information management system).

As used herein, “providing access to at least a portion of a database” refers to making information in the database available to user(s) through a visual or auditory means of communication.

The term “assessing” and “evaluating” are used interchangeably to refer to any form of measurement, and includes determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

The term “using” has its conventional meaning, and, as such, means employing, e.g. putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

Peptides selected from complex samples using methods of the present invention are enriched for methionine-, cysteine, and histidine-containing peptides, and can be concentrated when released from the Pd-substrate under eluting conditions.

Typical methods for concentrating a dilute peptide solution including adding several volumes of miscible organic solvent to effect precipitation, lyophilization (or “speed-Vac”) of the solution to remove aqueous or organic solvents by vapor pressure, adsorption to a solid-phase matrix, having either non-polar (reversed-phase) or polar (ion-exchange or normal-phase) characters, permitting elution of peptide with a small volume of suitable mobile phase, or removal of excess solvent by filtration through a semi-permeable membrane device, which permits the comparably larger peptides to be captured, while permitting flow of smaller solvent molecules, driven either by pressure, or by centrifugal force.

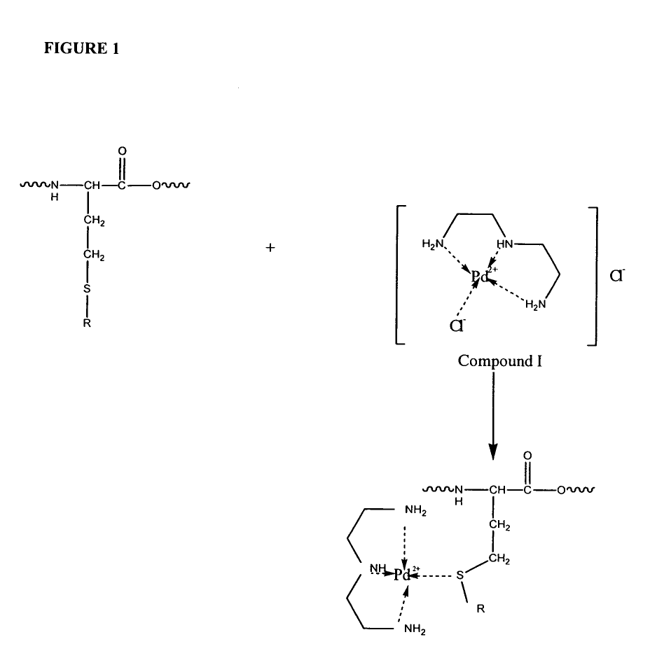

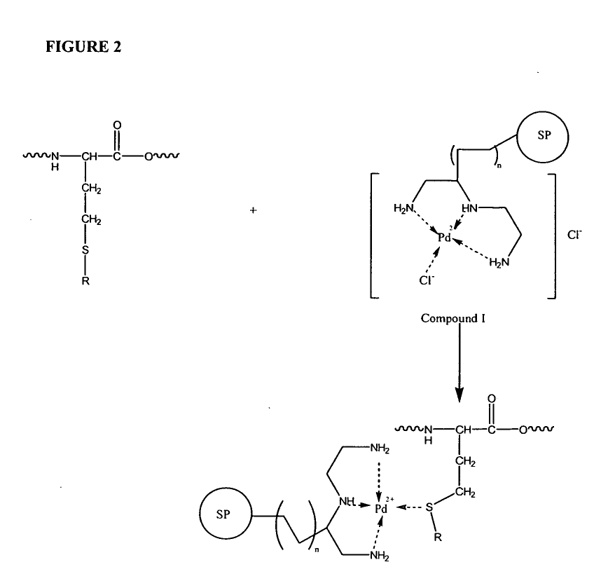

The Pd coordination compounds may be used to affinity purify biomolecules, such as proteins or peptides, comprising a sulfur (S) or nitrogen (N) group in a sample. See, e.g., as shown in FIG. 1. Thus, a Pd coordination compounds (such as shown in FIG. 2) may be used to reduce the complexity of a complex sample of proteins or peptides, such as a proteome or peptidome, by selectively binding to sulfur-containing or nitrogen-containing biomolecules (e.g., such as proteins/peptides comprising methionine-, histidine-, and reduced cysteine-containing proteins/peptides). Methionine occurs in the human proteome at a rate of 2.4%, and when combined with the cysteines present, the number of peptides per protein that can be selected for analysis should be nearly 3 times greater than for the ICAT reagent. Histidine occurs in the human proteome at a rate of about 2.2%. This increase in protein coverage may be used to provide additional confidence in protein identification, to improve the accuracy of relative quantification, and to detect post-translational modifications that may be present on the selected peptides.

An affinity-purified population of proteins may comprise natural proteins, synthetic proteins, modified proteins, unmodified proteins, processed proteins or unprocessed proteins, and combinations thereof. Although the invention describes analysis of proteins and peptides, the analysis of other molecules is also envisioned, for example, the substrate may be used to select nucleic acids, lipids, fatty acids and steroids for further analysis, such as in mass spectrometric techniques.

In one aspect, the method comprises the step of providing a sample comprising a plurality of proteins, contacting the sample with a Pd coordination compound with affinity for sulfur groups (e.g., such as proteins comprising methionine and reduced cysteine groups) and nitrogen groups (e.g., such as histidine), and eluting proteins bound to the Pd coordination compound, to obtain a population of proteins which are enriched for proteins comprising such groups. In one aspect, the sample comprises the proteome of a cell. The sample may be derived from a biological fluid, tissue, population of cells, biopsy, archival sample, environmental sample and the like.

In some aspects, proteins are cleaved into smaller fragments (e.g., peptides), before, during, or after contacting the proteins with the Pd coordination compound. In one aspect, proteins are contacted with one or more cleaving agents to produce peptide fragments having carboxy-terminal lysine or arginine residues. For example, proteins may be treated with trypsin, Lys-C, another protease, or any combination thereof.

Proteins subjected to affinity purification may be characterized using any technique used for protein analysis. For example, in one aspect, affinity-purified proteins or peptide fragments thereof are characterized using mass spectroscopy techniques. Proteins contacted with a cleaving agent, before, during or after contacting with a Pd coordination compound, generate peptide fragments that may be characterized by multistage or tandem mass. Where proteins are eluted, these may be contacted with cleaving agents. When peptides are eluted, in certain aspects, these are evaluated by mass spectrometry. In one aspect, the mass of a peptide determined by mass spectrometry is compared to the mass of a known or previously characterized peptide that may be correlated with sequence information for the known or previously characterized peptide. In one embodiment, the method further comprises searching one or more sequence databases for the sequence(s) observed for the protein or peptide fragment thereof.

In other aspects the affinity-purified proteins/peptides may be characterized using techniques such as SDS-PAGE, 2-D gel electrophoresis, isoelectric focusing, immunologically-based methods (e.g., western blotting and immunoprecipitation) and Edmund degradation.

In one embodiment, at least one coordination site of the palladium compound binds directly or indirectly (e.g., through a linker) to a substrate. As used herein, a “linker” refers to a bifunctional chemical moiety which comprises an end for stably associating with a substrate and an end for stably associating with the Pd-coordination compound (e.g., able to donate electrons to a coordination site of the Pd coordination compound).

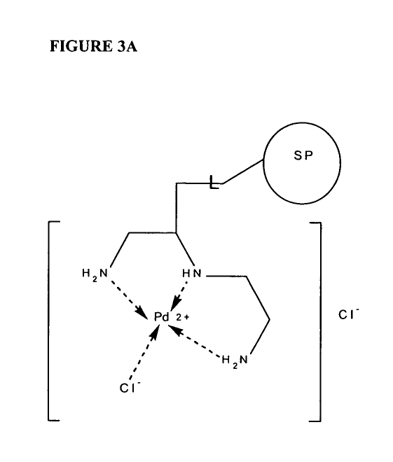

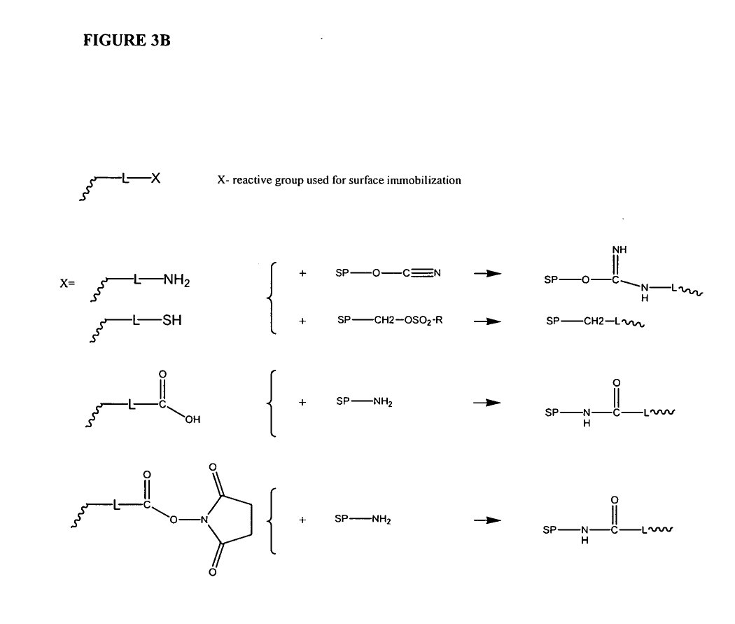

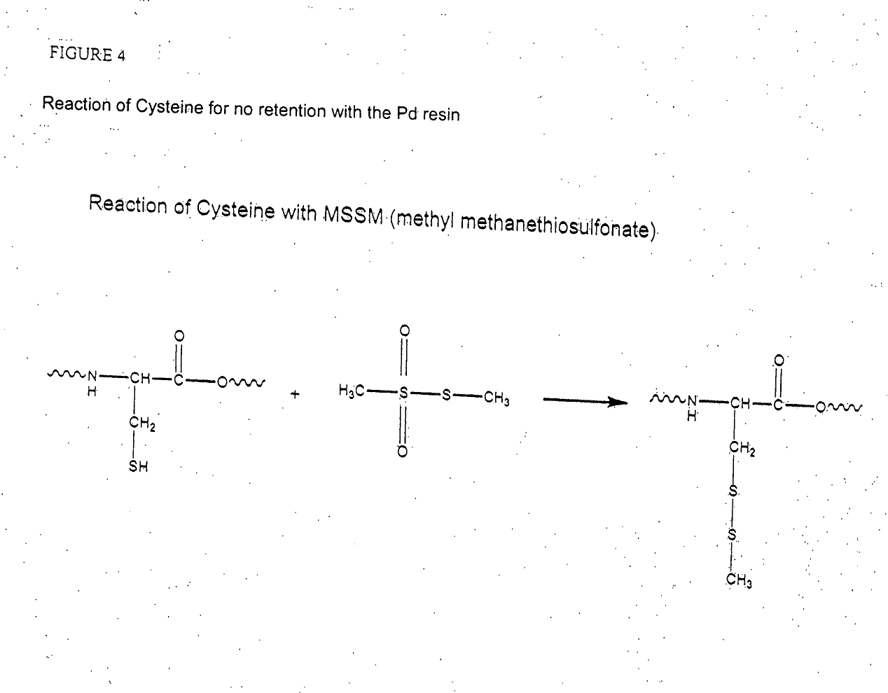

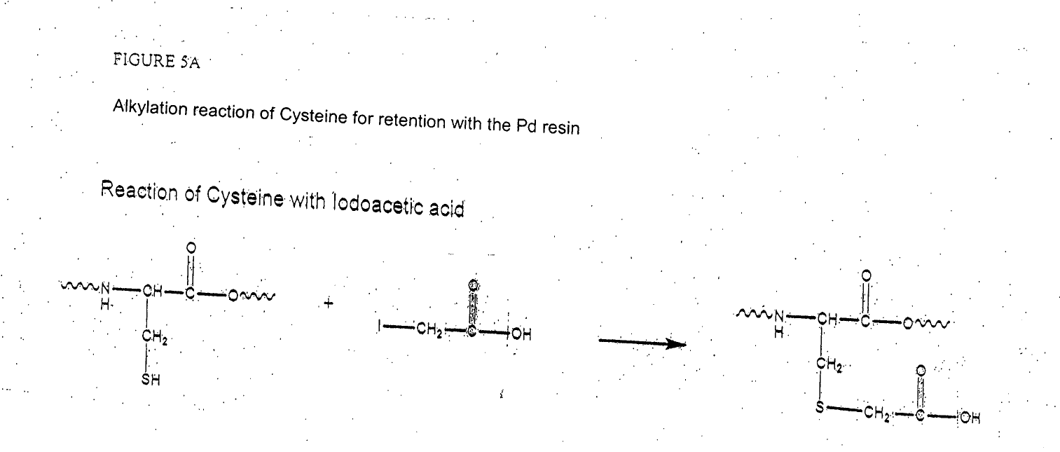

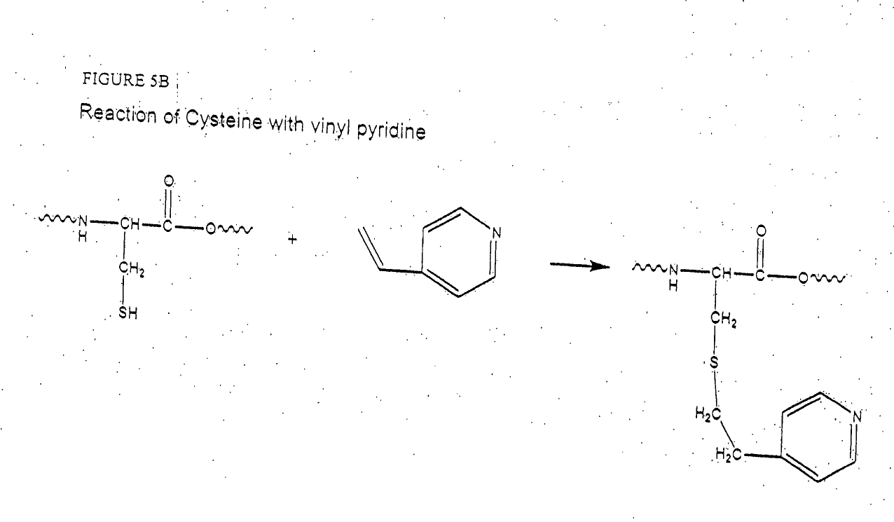

A linker, when other than a bond, will have from about 1 to 60 atoms, usually 1 to 30 atoms, where the atoms include C, N, O, S, P, etc., and will generally have from about 1 to 12 carbon atoms and may have from about 0 to 8 or more, or about 0 to 6 or more heteroatoms. The atoms are exclusive of hydrogen in referring to the number of atoms in a group, unless indicated otherwise. Additional types of linker molecules are described in, e.g., Backes and Ellman (1997) Curr. Opin. Chem. Biol. 1:86-93, Backes et al. (1996), J. Amer. Chem. Soc. 118:3055-3056, Backes and Ellman (1994), J. Amer. Chem. Soc. 116: 11171-11172, Hoffmann and Frank (1994), Tetrahedron Lett. 35:7763-7766, Kocis et al. (1993), Tetrahedron Lett. 34:7251-7252, and Plunkett and Ellman (1995), J. Org. Chem. 60:6006-6007. In one aspect, a linker comprises a group that may undergo a nucleophilic attack, including, but not limited to an ether, carboxylate, succinimide, or other like group. In one aspect, linkers are selected from the group shown in FIGS. 3A and 3B. Other linkers may include oligonucleotides or peptides (e.g., such as polylysine). In certain embodiments, linkers are cleavable from the substrate, e.g., they may include photocleavable groups, pH sensitive groups, thermolabile groups or sites for enzyme cleavage. In one aspect, linkers are selected which do not alter the ability of the Pd-compound to bind to other ligands. In another aspect, linkers may be selected which exhibit the least amount of non-selective binding to sample, e.g., linkers are inert to the sample components.

The linker may be covalently, ionically, or otherwise stably associated with the substrate. Stable associations can include covalent or non-covalent bonds and, and may be direct (i.e., the Pd coordination compound may bind to the substrate via an interaction between a coordination site on the Pd compound and a chemical group or molecule immobilized on the substrate) or indirect (i.e., the Pd compound may bind to a ligand that may bind covalently or non-covalently to a linker molecule which itself forms direct stable associations with the substrate). In a preferred embodiment the linker is covalently immobilized to the substrate.

In certain aspects of the invention, a Pd compound stably associated with a substrate (“Pd-substrate”) selectively binds to at least about one biomolecule. In one aspect, a biomolecule ligand coordinating the Pd portion of a Pd-substrate composition comprises a suitable electron density for donating to an empty orbital of the Pd group via a sulfur or nitrogen group.

In certain aspects, the Pd compound may include one or more stabilizing substituents, which are at least substantially stable or unreactive under conditions of storage and/or use of the substrate. The stabilizing substituents may be same or different from one another, and may be selected based upon the desired conditions of use since these substituents may affect the physical and/or chemical properties of the substrate, e.g., hydrophobicity/hydrophilicity, etc. In some aspects, stabilizing substituents are interconnected, together constituting a stabilizing bridge moiety. In one aspect, the stabilizing bridge is at least divalent, occupying two ligand sites with the palladium atom, but may be multivalent, occupying more than two such ligand sites. Aliphatic amine compounds may be used to form stabilizing bridges. In one aspect (e.g., when the Pd(II)-based are used) ethylenediamine is used to provide a divalent stabilizing bridge.

The use of a ligand that allows the Pd to be able to bind to two ligands of the peptide will result in a stronger binding and may result in less non-specific binding. In this case the Pd complex may be a diaminoethane rather than the triaminodiethylene compound shown in the examples. The triamine complex may associate to form a relatively weaker complex with the bound peptide and therefore result in a lower affinity binding solid phase.

Substrates may be provided in a variety of shapes and forms. For example, suitable substrates include, but are not limited to, gels, fibers, microspheres, spheres, cubes, particles, beads (including porous beads), pellets, planar substrates (e.g., slides, discs, wafers, chips), channels, microchannels, nanochannels, capillaries, walls of containers, membranes, webs, gels, sheets, tubing, spheres, containers, pads, slices, films, plates, slides, strips, plates, disks, rods, particles, beads, and filters.

The substrate may be formed of a variety of materials and the size and shape of the substrate is not a limiting feature of the invention. The substrate may be rigid or flexible or semi-flexible. The term “rigid” is used herein to refer to a structure e.g., a bottom surface that does not readily bend without breakage, i.e., the structure is not flexible. The term “flexible” is used herein to refer to a structure, e.g., a bottom surface or a cover, which is capable of being bent, folded or similarly manipulated without breakage. In one aspect, the substrate comprises a flexible web that can be bent 180 degrees around a roller of less than 1.25 cm in radius at a temperature of 20° C.

Rigid solid supports may be made from silicon, glass, rigid plastics, e.g. polytetrafluoroethylene, polypropylene, polystyrene, polycarbonate, etc., or metals, e.g. gold, palladium, etc. Flexible solid supports may be made from a variety of materials, such as, for example, nylon, nitrocellulose, polypropylene, polyester films, e.g., polyethylene terephthalate, polymethyl methacrylate or other acrylics, polyvinyl chloride or other vinyl resin. Various plasticizers and modifiers may be used with polymeric substrate materials to achieve selected flexibility characteristics.

In certain aspects, substrates are selected which may be conveniently sorted, e.g., facilitating collection of affinity-purified biomolecules. For example, substrates may be magnetic or magnetizable, such that exposure to a magnetic field may be used to sort or separate Pd-bound substrates complexed to biomolecules or substrates from which Pd-biomolecules have been removed, or Pd-bound substrates from which biomolecules have been removed.

In still other aspects, the Pd-substrate comprises a label or identifier (e.g., such as a bar code or radio frequency tag) identifying a sample or a container from which the sample is derived.

In one embodiment, substrate materials are selected which are particularly suited for interfacing with mass spectrophotometers. For example, in one aspect, the solid substrate comprises a chromatographic material, which under specified conditions binds all proteins or peptides comprising sulfur groups (e.g., such as proteins or peptides comprising a methionine or reduced cysteine) and proteins comprising nitrogen groups comprising electrons which can donate to a Pd coordination site, e.g., such as histidine. In certain embodiments, a substrate according to the invention is suitable for use in SELDI analysis.

In another aspect, the substrate comprises a web. As used herein, a “web” refers to a long continuous piece of substrate material having a length greater than a width. For example, the web length to width ratio may be at least 5/1, 10/1, 50/1, 100/1, 200/1, or 500/1, or even at least 1000/1. In one aspect, the web is flexible and may be spooled past various processing stations, i.e., stations comprising cleaving agents, wash buffers, elution solutions, and the like.

In a further aspect, the solid substrate comprises a material suitable for use in liquid chromatography, for example, a resin. As used herein, a “resin” refers to an insoluble material (e.g., a polymeric material) or particle that allows ready separation from liquid phase materials by filtration. In further aspects, resins may be used for the packing of chromatographic columns. Resins can be used to carry tags and/or tagged peptides. Suitable resins include, but are not limited to, agarose, guaracrylamide, silica based materials, carbon-based materials, carbohydrate-based polymers (e.g., polysaccharide-containing), and the like. In one embodiment the Pd-substrate composition is coupled to or packed into a column.

In one embodiment, the invention provides a method of using Pd-substrate compositions according to the invention for affinity purifying biomolecules comprising sulfur groups or nitrogen groups. In one aspect, the Pd-substrate compositions are used for affinity purifying proteins or peptides comprising cysteines, methionines, and histidines, and modified, processed or derivatized forms thereof. In one aspect, because the Pd-substrate will not react with the non-reduced cysteines, i.e. disulfide bonds, a protein sample is reduced prior to or while contacting with the Pd-substrate.

In one aspect, the method comprises the step of providing a sample comprising a plurality of proteins, contacting the sample with a substrate comprising compounds with affinity for sulfur groups (e.g., such as proteins comprising methionine and reduced cysteine groups) and nitrogen groups comprising electrons which can bind to Pd coordination sites (e.g., such as histidine), and eluting proteins bound to the substrate, to obtain a population of proteins which are enriched for proteins comprising such groups. Suitable buffers for binding include those that do not comprise components or atoms that would bind to Pd coordination sites.

Samples may be complex samples (e.g., comprising proteomes or peptidomes) or may be samples that have been subjected to one or more previous purification steps. For example samples may be treated prior to contacting with Pd-substrate columns to remove high abundance proteins such as albumin. Purification may be based on hydrophobicity, size, charge, sequence, and the like, and may be performed using known methods such as SDS-gel electrophoresis, size-exclusion chromatography, isoelectric focusing, capillary electrophoresis and the like. Additionally, or alternatively, small volumes of sample may be concentrated prior to contacting with Pd-substrate compositions. In certain aspects, non-protein and non-peptide components are removed from the sample.

In one embodiment, Pd-substrate compositions are used to analyze a complex protein sample comprising at least about 20% of total protein coming from a biological sample source, usually at least about 40%, more usually at least about 75%, and generally 90% or more, up to and including all of the protein obtainable from the source. The proteome may be present in a cell, a lysate, a microsomal fraction, an organelle, a partially extracted lysate, biological fluid, and the like. The proteome will be a mixture of proteins, generally having at least about 100 different proteins, usually at least about 1000 different proteins and in most cases, about 5,000 different proteins or more.

Generally, the sample will have at least about 0.05 mg of protein, usually at least about 1 mg of protein or about 10 mg of protein or more, typically at a concentration in the range of about 0.1-20 mg/ml, preferably, about 0.5-2.0 mg/ml, and most preferably about 1.0-2.0 mg/ml. The sample may be adjusted to the appropriate buffer concentration and pH.

In one embodiment, as shown in FIG. 1, Pd-substrate compositions according to the invention bind to proteins/peptides in a manner that is reversible, i.e., when treated with an appropriate liquid-phase reagent (eluting condition), the proteins/peptides will desorb from the Pd-substrate. Suitable eluting conditions include exposure to solutions comprising beta-mercaptoethanol, sodium bisulfite, molecules comprising or capable of forming CN⁻ groups, thiocyanates (e.g., potassium thiocyanate, guanidinethiocyanate), sodium thiosulphate, sodium metabisulphite, aromatic or aryl cyanides, or a molecule which competes with the bound biomolecule for the Pd coordination site, and the like.

The reduced cysteines can be protected (or blocked) with an appropriate alkylating reagent, such as iodoacetic acid or vinyl pyridine.

In some aspects, a population of proteins is contacted with a cleaving agent before, after, or during exposure to a cleaving agent. In one aspect, the proteins are exposed to a cleaving agent before, during, or after binding to a Pd-substrate composition but prior to elution of the proteins from the column. In this way, a population of peptides bound to the Pd-substrate compositions may be generated or an eluted population of peptides may be generated. Such peptides may comprise at least one methionine, cysteine, or histidine. Methionine-containing peptides may represent N-terminal fragments of proteins.

The peptides that do not contain a methionine or a reduced cysteine can be specifically selected and removed, leaving the Met and Cys peptides bound to the substrate.

In certain embodiments, it is preferred that peptides comprising methionine, cysteine, and histidine are selected.

In other embodiments, it is preferred that peptides comprising methionine and histidine are selected. In such instances the cysteines of the peptides are reacted with a reagent which forms disulfide bonds with cysteine, such as a methanethiosulfonate (MTS) reagent (e.g., allyl methanethiosulfonate, 2-(aminocarbonyl)ethyl methanethiosulfonate, 2-(4-aminobenzoyloxy)ethyl methanethiosulfonate, benzyl methanethiosulfonate, butyl methanethiosulfonate, 2-carboxyethyl methanethiosulfonate, decyl methanethiosulfonate, dodecyl methanethiosulfonate, N-(β-D-glucopyranosyl)-N′-[(2-methanethiosulfonyl)ethyl]urea, hexadecyl methanethiosulfonate, 2-hydroxyethyl methanethiosulfonate, 6-hydroxyhexyl methanethiosulfonate, O-2-(methanethiosulfonyl)ethyl N-[2-(N,N-dimethylamino)ethyl]carbamate, 3-methanethiosulfonyl-N,N-dimethylpropionamide, methoxypoly(ethylene glycol)-5000-succinamidoethyl methanethiosulfonate, methyl methanethiolsulfonate (MSSM), octyl methanethiosulfonate, pentyl methanethiosulfonate, propyl methanethiosulfonate, and pyridinedithioethylamine), in order to prevent cysteine binding to the Pd-substrate.

Selection of His-only peptides can be achieved by passing the peptide mixture over a suitable resin to remove all the Cys and Met containing peptides and then passed over the Pd-substrate to bind the His peptides.

Suitable cleaving agents include, but are not limited to, enzymes, for example, one or more of: serine proteases (e.g., such as trypsin, hepsin, SCCE, TADG12, TADG14); metalloproteases (e.g., such as PUMP-1); chymotrypsin; cathepsin; pepsin; elastase; pronase; Arg-C; Asp-N; Glu-C; Lys-C; carboxypeptidases A, B, and/or C; dispase; thermolysin; cysteine proteases such as gingipains, TEV protease, factor Xa and the like. Proteases may be isolated from cells or obtained through recombinant techniques. The cleaving agent is not limited to an enzyme and can be a chemical reagent, for example, cyanogen bromide (CNBr), 2-nitro-5-thiocyanobenzoic acid, N-bromosuccinamide and other reactive halogen compounds, hydroxylamine, 1-2M formic or acetic acid, periodate oxidation, 2-(2-nitrophenylsulfenyl)-3-methyl-3-bromoindolenine or o-iodosobenzoic acid (See, for example, Hermodson et al., “Methods in Protein Sequence Analysis”, ed. Elzinga, Humana Press, Clifton, N.J., pp. 313-323, 1982).

In some aspects, the cleaving agent may be associated with the substrate. For example, the cleaving agent may be disposed within the pores of substrates comprising porous beads or may be immobilized via a binding partner.

Proteins or peptide fragments thereof subjected to affinity purification may be characterized using a variety of techniques. Affinity-purified proteins may be analyzed using a variety of techniques such as by mass spectrometry, including, but not limited to MALDI-TOF-MS, SELDI-TOF-MS, ESI, TOF, ion trap mass spectrometry, ion trap/TOF mass spectrometry, quadropole mass spectrometry, Fourier Transform mass spectrometry, fast atomic bombardment (FAB), plasma desorption (PD), thermospray (TS), and magnetic sector mass spectrometry.

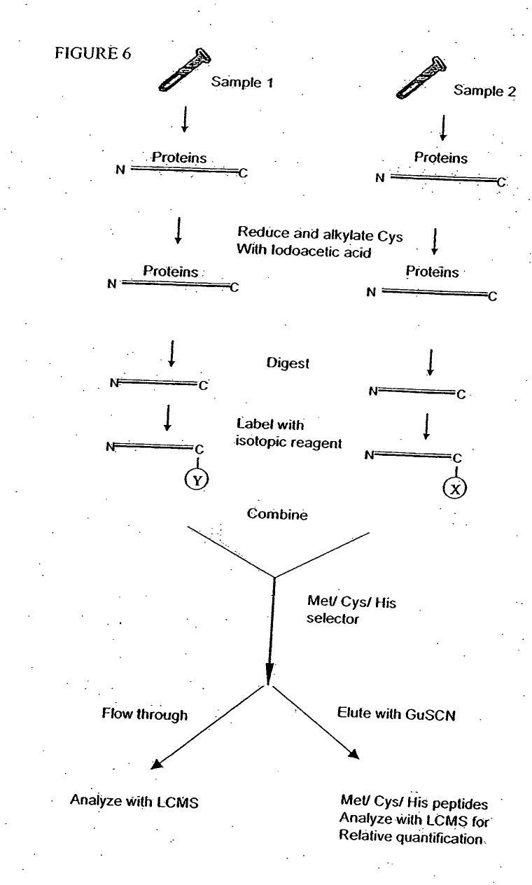

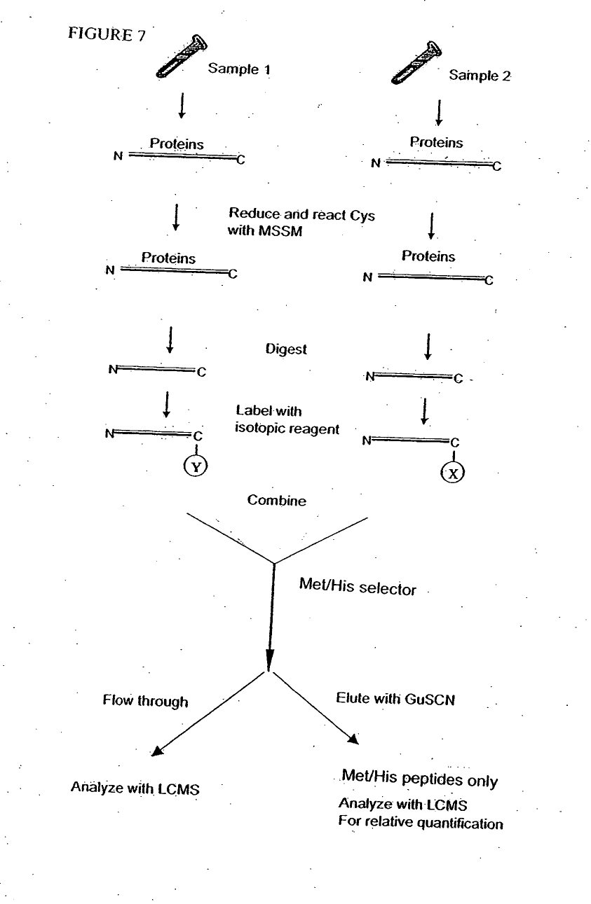

In one aspect, e.g., for differential analysis, at least two samples are subjected to affinity purification using Pd-substrate compositions according to the invention. The samples are differentially labeled, e.g., each protein sample comprises a different label or one sample is labeled while the other is unlabeled. In one aspect, the label is a mass-altering label. The samples may be mixed before or after affinity purification (e.g., see FIGS. 6-8).

In one aspect, the sum of the masses of the constituent atoms of the label is preferably uniquely different than the fragments of all the possible amino acids. As a result, labeled peptides and labeled fragments are readily distinguished from unlabeled peptides/unlabeled fragments by their ion/mass pattern in the resulting mass spectrum. In one aspect, the label does not suppress the ionization efficiency of the peptide. In another aspect, the label remains soluble in an MS buffer system of choice. In another aspect, peptides are labeled is such a way that the label will bind selectively to the Pd-substrate.

Suitable mass-altering labels include stable isotopes, including but not limited to isotopes of hydrogen, nitrogen, oxygen, carbon, or sulfur, such as, ²H, ¹³C, ¹⁵N, ¹⁷O, ¹⁸O, or ³⁴S, and combinations thereof. In certain embodiments, pairs of stable isotopes are used which provide heavy and light mass labels. Such pairs include, but are not limited to H and D, ¹⁶O and ¹⁸O, ¹⁶O and ¹⁷O, ²C and ¹³C, ¹⁴N and ¹⁵N, ³² S and ³⁴S. In certain embodiments, the mass-altering label does not comprise palladium.

In another aspect, the mass-altering label is part of a peptide comprising a modification, i.e., peptides comprising the modification and peptides lacking the modification are distinguishable by mass. The modification may comprise a phosphorylated residue, a glycosylated residue, an acetylated residue, a ubiquitinated residue, a ribosylated residue, methylated residue, a sulfated residue, a prenylated residue, a hydroxylated residue, or a farnesylated residue.

In one aspect, the mass of a labeled peptide is determined and correlated with the identity and/or activity of a protein (e.g., the presence of a particular modified form of a protein which is known to be active in the proteome being evaluated). Preferably, a mass-to-charge ratio is determined, e.g., by mass spectrometry. In addition to determining the identity of a protein, a quantitative measure of the amount of protein in the sample may be obtained. In certain aspects, the site of a modification may be determined by reacting sample proteins or peptides with a label comprising a reactive site which reacts with a modified residue on the protein. Similarly, the relative amount of a modified protein compared to unmodified protein also may be determined.

When comparing several samples, one sample may be used as a reference sample, to which other samples are related. In one aspect, after differential labeling of samples, samples are combined (generally, equal amounts of samples are combined) and contacted with Pd-substrate compositions according to the invention. In one aspect, proteins bound to the Pd-substrate are exposed to a cleaving agent (e.g., such as trypsin) to generate peptide-Pd-substrate complexes. In another aspect, peptides are eluted from the Pd-substrate under eluting conditions (e.g., such as exposure to a solution comprising molecules comprising cyanates, beta mercaptoethanol, sodium bisulfite, sodium sulfite, potassium thiocyanate, guanidinethiocyanate, sodium thiosulphate, sodium metabisulphite, aromatic or aryl cyanides and the like).

Eluted peptides may be subjected to one or more separation steps. Such separations may be based on size, charge, hydrophobicity or a combination thereof to obtain purified peptides whose mass may be determined (e.g., after fragmentation in a mass spectrometer). Suitable separation techniques include, but are not limited to: High Pressure Liquid Chromatography (HPLC), Low Pressure Liquid Chromatography, Reverse Phase-High Pressure Liquid Chromatography (RP-HPLC), gel electrophoresis (including capillary gel electrophoresis or any other electrophoretic modes), cation or anion exchange chromatography, or any of a number of peptide purification methods as are known in the art. In some aspects, separation is performed using a device which may be interfaced to a mass spectrometer. For example, separations may be performed using microcapillary liquid chromatography.

The mass of a peptide selected using Pd-substrates according to the invention may be determined and correlated with the identity and/or activity of a protein (e.g., the presence of a particular modified form of a protein which is known to be active). Preferably, a mass-to-charge ratio is determined, e.g., by multistage mass spectrometric analysis. In addition to determining the identity of a protein, a quantitative measure of the amount of protein in the sample may be obtained. The method may also be used to determine the site of a modification of a protein in one or more samples, by reacting sample proteins with a tag molecule comprising a reactive site that reacts with a modified residue on the protein. In another aspect, the amount of a modified protein in a sample is also determined.

Peptide sequence information may be automatically generated by selecting peptide ions of a particular mass-to-charge (m/z) ratio for collision-induced dissociation (CID) or other means for generating peptide ions known in the art. The resulting ionization spectra may then be correlated with sequences in sequence databases to identify the protein from which the sequenced peptide originated, e.g., using computer searching algorithms known in the art.

Peptides may be quantified by measuring the relative signal intensities for pairs of peptide ions of identical sequence that are tagged using different mass-altering labels, e.g., such as light or heavy forms of isotope, or which comprise label and unlabeled peptide pairs (which differ in mass by the mass of the label). In certain aspects, a peptide or mass-altering portion of a peptide may comprise a detectable label such as a radioactive label, spin label, chemiluminescent label, and the like.

In some aspects, mass spectrometry analysis may be used to determine both the quantity and identity of proteins from which labeled peptides are derived, for example, by using an automated multistage mass spectrometer and alternating scans which measure quantities of peptides eluting from a separation column and record sequence information from selected peptides.

In one embodiment, methods according to the invention are used to evaluate samples which have been exposed to an agent. A reference sample may comprise a sample which is not exposed to the agent. Additional samples may comprise samples exposed to different concentrations of agents. Suitable agents which can be evaluated include, but are not limited to: drugs; toxins; proteins; proteins; peptides; amino acids; antigens; cells, cell nuclei, organelles, portions of cell membranes; viruses; receptors; modulators of receptors (e.g., agonists, antagonists, and the like); enzymes; enzyme modulators (e.g., such as inhibitors, cofactors, and the like); enzyme substrates; hormones; nucleic acids (e.g., such as oligonucleotides; polynucleotides; genes, cDNAs; RNA; antisense molecules, ribozymes, aptamers), and combinations thereof. Agents also can be obtained from synthetic libraries which are commercially available or generated through combinatorial synthesis using methods well known in the art.

Agents associated with a desired cell state or the transition from an undesired cell state (e.g., a pathology) to a desired cell state (e.g., absence of the pathology or reduction in symptoms or biomarkers diagnostic of the pathology) may be identified as candidate compounds for treating the undesired cell state, for example, in a patient from whom the sample of cells was derived. Such compounds may be formulated as pharmaceutical compositions using methods known in the art.

In certain aspects, expression of a protein or set or proteins, and/or modified and/or cleaved forms thereof, associated with a particular cell state may be used to generate diagnostic probes to detect or screen for the cell states. Such proteins (or modified or cleaved forms) may be detected directly, e.g., using mass spectrometry techniques or indirectly, e.g., using antibody probes.

In one aspect the invention relates to a system comprising a Pd coordination compound and an analysis system for determining at least one characteristic of a protein or peptide separated or recovered from the Pd coordination compound. In certain embodiments the Pd coordination compound is stably associated with a substrate to forma a Pd-substrate composition.

In one embodiment, the invention further provides a system which interfaces Pd-substrate compositions with a protein analysis system such as a mass spectrometer. In one aspect, the system comprises a Pd-substrate composition directly or indirectly coupled to a separation device such as a column, capillary, or a channel (e.g., a microchannel or nanochannel) in a substrate. The palladium-substrate may comprise a Pd-resin which is itself disposed in a column, capillary, or channel and which communicates, directly or indirectly with the separation device. In certain embodiments, where the Pd-substrate is a planar substrate, the substrate may be part of or contained within a chamber and peptides eluted from the substrate may be collected via a tube, capillary, spray, or injection device for delivery to the separation device. In one aspect, the separation device may be an HPLC device, RP-HPLC device, an LC-microcapillary device, a gel matrix, an ion exchange matrix, and the like. The separation device may function to separate peptides, purify individual peptides, and concentrate purified peptides. Both the Pd-substrate and separation device may be contained within a micro-scale or nano-scale device. In one aspect, Pd-substrates and separation devices are separated by channels or reservoirs which may be used to add or exchange buffers or elution solutions used for affinity purification for buffers or other solutions suitable for separation.

The pH of the eluting solvent can be normally set within the range from 5.0 to 9.0. The column temperature is normally set within the range from about 20 to 40° C.

The proper column size will be readily ascertainable to those skilled in the art and can be appropriately set depending on the amount, concentration, purity, etc. of the liquid sample used. For example, the column may have outer diameters that range from about 0.1 mm to about 50 mm and lengths up to about 300 mm. In the case of using high performance liquid chromatography, the flow rate of a column mobile phase can be normally set within the range from 0.5 to 1.5 mL/min., when employing a column of about 5 mm ID. Typical flow rates will increase or decrease proportionately with the internal diameter of the column, in a manner well known to those skilled in the art. A column could be any type of chromatography column, including an analytical column, a preparatory column or a guard column, may comprise micro-fluidic devices formed from etched polymers (such as the HPLC Chip manufactured by Agilent Technologies Inc.) and may be formed from various materials such as fused silica, stainless steel, glass lined stainless steel, or stainless steel capillary lined with coated fused silica. A feature of the materials so employed is that they will preferably not adsorb peptides on their surfaces (are thereby inert towards the sample).

In another embodiment, the invention provides a computer program product comprising a computer readable medium comprising instructions for controlling functions of a system described above.

The invention additionally provides computer program products comprising computer readable medium providing instructions to a processor in communication with a system described above to control one or more system functions, e.g., exposure of Pd-substrate compositions to elution conditions, contacting proteins with a cleaving agent, ionization or peptide fragments, delivery of peptides to a mass spectrometer, and analysis of mass spectra.

The invention additionally provides a system comprising a computer readable medium comprising a memory, wherein the memory comprises mass spectral data relating to a plurality of peptides, wherein each peptide comprises at least one cysteine, methionine, and/or histidine residue. In certain embodiments the system comprises data relating to the quantity of a peptide in the sample.

An embodiment of the invention also includes forwarding, communicating, or receiving data produced from any method of the invention. Another embodiment can additionally include forwarding a sample to a remote location and receiving data communicated from the remote location using that sample in a method of the present invention.

In another embodiment, the system comprises a computer readable medium comprising a memory, wherein the memory comprises mass spectral data relating to a plurality of peptides, wherein each peptide comprises at least one cysteine, methionine, and/or histidine residue. The memory may additionally comprise data relating to the mass of a peptide in a sample, the type of sample, data relating to agents to which the sample has been exposed, data relating to an organism (e.g., such as a human patient) from which a sample has been derived (e.g., such as medical history, drug exposure, and the like).

In one aspect, the separation module and or affinity purification composition may be in communication with a detector. In such aspect, peptides or their mass-altering tags when such are used may be labeled with a label detectable by the detector (e.g., such as a radioactive label, spin label, chemiluminescent label, and the like).

In some aspects, throughput may be increased by dividing eluted peptides into a plurality of sets and separating the sets using a plurality of separation devices operating in parallel and/or in series.

In one aspect, the separation module interfaces with an mass spectrometer device through an interfacing module (such as an electrospray device, such as an electrospray capillary or nozzle) which delivers substantially purified peptides comprising, methionines, histidines, reduced cysteines, or other sulfur or nitrogen groups (e.g., from derivatizing agents) comprising electron groups which may bind to coordination sites of Pd compounds (e.g., generated by derivatizing the peptides with one or more chemical moieties) to the mass spectrometer. In one aspect, for example, phosphorylated residues may be tagged with a derivatizing agent which comprises or generates a sulfur or nitrogen group suitable for binding to Pd coordination sites. In the case where the protein analysis system comprises a MALDI device, an automated spotter may be used to connect a separation capillary to a MALDI device (see, e.g., Figeys et al., 1998, Electrophoresis 19: 2338-2347).

In another aspect, the separation module interfaces with the Pd-coordination compound or Pd-substrate composition.

Throughput of the delivery process may be increased using arrays of electrospray or nanospray needles. (See, e.g., Zubritsky et al., 2000, Anal. Chem. 72: 22A; Licklider et al., Anal. Chem. 72: 367-375; Scherer et al., 1999, Electrophoresis 20: 1508-1517).

Fluids may be moved through the system using mechanisms known in the art such as pressure or electro-osmotic pumping.

In another aspect, the system comprises one or more detectors for detecting movements of fluids, proteins, and/or peptides through the system.

Generally, the mass spectrometer device of the system comprises an ionizer, an ion analyzer and a detector.

Any ionizer that is capable of producing ionized peptides in the gas phase can be used, such as an ion spray mass spectrometer (Bruins et al., 1987, Anal Chem. 59: 2642-2647), an electrospray mass spectrometer (Fenn et al., 1989, Science 246: 64-71), and laser desorption device (including matrix-assisted desorption ionization and surfaced enhanced desorption ionization devices). Any appropriate ion analyzer can be used as well, including, but not limited to, quadropole mass filters, ion-traps, magnetic sectors, time-of-flight, and Fourier Transform Ion Cyclotron Resonance (FTICR). In a preferred aspect, a tandem MS instrument such as a triple quadropole, ion-trap, quadropole-time-of flight, ion-trap-time of flight, or an FTICR is used to provide ion spectra. A FAB ionizer may also be used.

In one aspect, molecular ions generated by ionization of peptides delivered, for example, from an electrospray or nanospray are accelerated through an ion analyzer as charged molecules. Ions generated may be detected using any suitable detector. In one aspect, ions are isolated and fragmented to generate daughter ions which when detected may provide a unique signature for the peptide.

Generally, peptides typically fragment at the amide bond between amino acid residues and peaks correspond to particular amino acids or combinations of amino acids. While there may be additional peaks (ions) present in the product ion spectra, many of these other peaks can be predicted and their presence explained by comparison with spectral data of known compounds (e.g., standards). Many different processes can be used to fragment the parent ion to form product ions, including, but not limited to, collision-induced dissociation (CID), electron capture dissociation, and post-source decay.

In another aspect, the system further comprises a system processor which can convert signals obtained from different components of the system (e.g., such as electrical signals) into data and can provide instructions for controlling one or more system functions. In one aspect, data includes, but is not limited to, data relating to an identifier on a Pd-substrate, data relating to binding conditions and/or elution conditions during affinity purification using the Pd-substrate, data relating to separation, concentration, and/or purification of peptides eluted from the Pd-substrate using the separation device, data relating to fluid movement in the system (e.g., the operation of pressure or electroosmotic pumps), as well as data relating to peptide fragmentation, ionization, peptide quantity and amino acid sequence.

In certain embodiment the system function is selected from the group consisting of contacting sample to a Pd-coordination compound associated with a substrate, eluting proteins or peptides bound to a Pd-coordination compound, contacting a sample with a cleaving agent, separating proteins and/or peptides, concentrating proteins and/or peptides, ionizing peptides, moving fluid through the system, and combinations thereof.

In some aspects, the processor compares mass spectral data to sequences in a protein and/or nucleic acid sequence database which the processor may access remotely. Thus, in a further aspect, the system further comprises a memory for storing data relating to peptide masses, and/or amino acid sequence. In another aspect, the system additionally comprises an information management system for searching and comparing data in the memory and obtained from mass spectrometry analysis. However, in other aspects, the processor obtains sequence information directly from mass spectral data provided to it from the mass spectrometer. The type of protein or peptide analysis performed by the system processor will relate to the type of mass spectrometer or other protein analysis device used in the system.

In still another aspect, in response to data from various system components, the processor alters one or more functions of the system. In additional or alternative embodiments, the processor is programmed e.g., by a user of the system and/or remotely to provide particular system instructions.

The invention additionally provides a computer program comprising a computer readable medium comprising instructions for controlling a system of the present invention. The invention additionally provides computer program products comprising computer readable medium providing instructions to a processor in communication with a system described above to control one or more system functions, e.g., exposure of Pd-substrate compositions to elution conditions, contacting proteins to a cleaving agent, ionization or peptide fragments, delivery of peptides to a mass spectrometer, and analysis of mass spectra.

In a further embodiment, the invention provides kits for facilitating methods according to embodiments of the invention. In one aspect, the kit comprises Pd coordination compounds stably associated with the substrate. In another aspect, the kit comprises a Pd coordination compound and a solid substrate, along with suitable reagents for stably associating the compound with the substrate. In still another aspect, the kit includes any combination of a number of solutions.

In one aspect, the kit comprises a suitable sample contacting solution for promoting binding between sample proteins and/or peptides to a Pd substrate. In one aspect, the solution promotes binding between proteins/peptides comprising sulfur or nitrogen groups, such as proteins comprising methionines, histidines, and/or reduced cysteines, or nitrogen or sulfur groups comprising electrons which may bind to Pd coordination agents, e.g., such as found on certain derivatized proteins. In one aspect, the kit further comprises derivatizing agents. In another aspect the kit comprises an identifier that comprises identifying information relating to a sample to be contacted with the coordination compound. In other aspects the Pd-substrate composition of the kit is coupled to or packed into a column.

The kit may additionally include a reagent for reducing di-sulfide bonded proteins and/or an alkylating agent. In still another aspect, the kit comprises an elution solution for removing a biomolecule bound to the Pd-coordination compound from the Pd-coordination compound. Such a solution may include, beta mercaptoethanol, sodium sulfite, sodium bisulfite, potassium thiocyanate, guanidinethiocyanate, sodium thiosulphate, sodium metabisulphite, aryl and aromatic cyanides, cyanates, molecules which may compete with biomolecules bound to the coordination sites of the Pd compound and the like. In a further aspect, the kit comprises a cleaving agent such as trypsin. Additional kit reagents may include labels, such as mass-altering labels, label pairs (such as heavy and light isotopes), reference biomolecules such as peptide standards or lock mass molecules, buffers and reagents for use in peptide separations such as HPLC, RP-HPLC, cation or anion exchange, electrophoresis, and/or buffers suitable for use in biomolecule analysis systems such as mass spectrometers.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The various aspects of the present invention are further described in the following non-limiting examples.

### EXAMPLES

### Example 1

### Conjugation of Palladium to Substrate

The palladium/linker complex is attached to the substrate by reacting a palladium/linker that is terminated by a reactive group with an activated substrate. The substrate comprises a reactive moiety while the palladium/linker comprises a reactive group. The reactive moiety is any organic or inorganic group which, under appropriate conditions or by the addition of suitable reagents, will react with the palladium/linker reactive group to form a bond so that a Pd-substrate composition is formed. The reactive moiety can include any reactive species, including a cyanato group (—O—C≡N), a sulfonic acid ester group (—CH₂—OSO₂-alkyl), or a primary amine group (—NH₂) (see FIG. 3B). These groups are attached directly onto the substrate, or they may be attached to a polyfunctional chemical moiety that is attached to the substrate. The polyfunctional chemical moiety is attached to the solid support and is also attached to the active site. The polyfunctional chemical moiety may be aliphatic, aromatic, alkyl, alkenyl, heteroaliphatic, heteroaromatic, any suitably reactive inorganic compound, or any combination thereof.

The reactive group on the linker is any organic or inorganic group that will react with the reactive moiety to form a bond so that a Pd-substrate composition is formed. The reactive groups are usually nucleophilic groups. Examples of reactive groups include a primary amine or —NH₂ group, a thiol group or —SH group, a carboxylic acid or —CO₂H group, or a carboxylic acid succinimide ester or —C(═O)—ONC₄H₄O₂ group.

The attachment of the palladium/linker complex to the substrate is performed by formation of an isourea linkage or —C(═NH₂)—NH— linkage. In this instance the reactive moiety on the substrate is a cyanato group, and the reactive group on the palladium/linker complex is a primary amine group or a primary thiol group. Such a reaction is performed under conditions that are well known in the art. The reaction conditions are selected such that the isourea linkage is formed without significantly degrading other parts of the molecule.

The attachment of the palladium/linker complex to the substrate is alternatively accomplished by forming an alkyl linkage. In this instance, the reactive moiety on the substrate is sulfonic acid ester or —CH₂—OSO₂—R, and the reactive group on the palladium/linker complex is a primary amine group or a primary thiol group. The organic group on the ester that is bonded to the sulfur is selected so that the sulfonic acid ester is a good leaving group to form an alkyl linkage between the palladium/linker complex and the solid substrate.

The attachment of the palladium/linker complex to the activated substrate is alternatively accomplished by forming an amide linkage. In one instance, the reactive moiety on the substrate is a primary amine or —NH₂, and the reactive group on the palladium-linker complex is a carboxylic acid group.

Reaction of the amine with the carboxylic acid to form an amide bond may be undertaken directly at any suitable condition. In cases where the reaction between the acid and the primary amine does not occur readily, it may be necessary to elevate the reaction temperature for the formation of the amide bond to occur. Alternatively, the reaction may proceed in good yield at room temperature by the use of coupling agents, such as dicyclohexylcarodiimide. Other exemplary agents include N,N′-carbonyldiimidazole, POCl₃, TiCl₄, sulfuryl chloride fluoride, chlorosulfonyl isocyanate, pyridinium salts-Bu₃N, or a mixture of Bu₃P and PhCNO.

Alternatively, the amide linkage may be formed by selecting a carboxylic acid succinimide ester as the reactive group on the palladium/linker complex.

### Example 2

### Preparation of Protein Samples

Samples containing a multiplicity of proteins can be obtained from any source. However, typically the proteins will be procured from tissue or cells or from body fluid such as plasma, serum, CSF (cerebrospinal fluid) and urine. Tissue is homogenized and/or into smaller groups of cells in order to facilitate lysis.

Cells are lysed via any of a number of protocols known in the art including physical disruption of cells, lysis in hypotonic solution, or lysis via ionic or non-ionic detergents. Following lysis, cell debris is removed via centrifugation and the supernatant containing the protein sample is collected. The concentration of protein is determined and the proteins are concentrated or diluted to a concentration in the range of about 0.1-20.0 mg/ml.

### Example 3

### Reaction of Cysteine Residues with methyl methanethiosulfonate (MSSM)

As noted above, the invention provides for selection of peptides or proteins comprising methionine alone; methionine and cysteine; methionine and histidine; and methionine, cysteine, and histidine.

In certain circumstances it will be useful to have peptides comprising cysteine residues not bind to the palladium. In such instances the protein sample is treated with methyl methanethiosulfonate (MSSM) in order to form a disulfide bond which will prevent interaction of the sulfur groups with the palladium.

Chemical modification with MSSM is carried out as described by Berglund et al. (1997) J. Am. Chem. Soc., 119: 5265-5255 and DeSantis et al. (1998) Biochemistry, 37: 5968-5973. Briefly, 200 μL of a 1 M solution of the MSSM reagent is added to a solution (5-10 mg/mL, 3.5 mL) of the protein sample in 70 mM CHES, 5 mM MES, 2 mM CaCl₂, pH 9.5. The MSSM reagent is added in two portions over 30 minutes. Reaction mixtures are kept at 20° C. with continuous end-over-end mixing. Reactions are monitored by following the specific activity (e.g. with suc-AAPF-pNA) and by tests for residual free thiol (e.g. with Ellman's reagent). Once the reaction is complete, the reaction mixture is loaded on a Sephadex™. (PD-10 G25 column with 5 mM MES and 2 mM CaCl₂, pH 6.5). Optionally, the protein fraction is then dialyzed against 1 mM CaCl₂ and the dialysate is lyophilized or suitable buffer exchange.

### Example 4

### Alkylation Reaction of Cysteine Residues for Retention with Pd-Substrate

In other circumstances it will be useful to have the cysteine residues of peptides protected in order to prevent the formation of disulfide bonds In such instances the protein sample is treated with iodoacetic acid or vinyl pyridine which will bond to the sulfur of the cysteine thus ensuring its availability to interact with the palladium.

The protein sample is prepared in 100 mM Tris pH 8.5. 10 μL 1M dithiothreitol is added and the reduction reaction proceeds for 2 hrs at ambient temperature. 20 μL 1M iodoacetic acid is added to the mixture and incubated for 30 min at ambient temperature in the dark. 40 μL 1M dithiothreitol is then added to quench the iodoacetic acid. The protein is then purified using dialysis, spin columns, or reverse phase chromatography or used “as is” for the nest step in the experiment.

Alternatively, the Cys sulfhydryl group is alkylated by reaction with 4-vinyl pyridine using the method originally devised by Friedman and coworkers (1970 J. Biol. Chem. 245, p. 3868-3871).

### Example 5

### Digestion of Samples to Generate Peptides

In certain instances, it is preferable that sample proteins are cleaved into smaller fragments (e.g., peptides), before, during, or after contacting the proteins with the Pd-substrate composition. Accordingly, proteins are contacted with one or more cleaving agents such as trypsin. Trypsin digestion is performed in buffer comprising 100 mM Tris-HCl (pH 8.5). The digestion is typically performed overnight at 37° C. in the dark.

### Example 6

### Labeling of Peptides with Isotopic Agent

For differential analysis, at least two samples are subjected to affinity purification using Pd-substrate compositions according to the invention. The samples are differentially labeled, e.g., each protein sample comprises a different label or one sample is labeled while the other is unlabeled. Relative quantitation between two proteomic samples is performed by derivatizing a specific side chain of a residue in the peptides or derivatizing the C- or N-terminal of peptides after an enzymatic digest. Such derivatizations include acylation of the N-terminus (Ji, J., Chakraborty, A., Geng, M. et al. (2000), ‘Strategy for qualitative and quantitative analysis in proteomics based on signature peptides’, J. Chromatogr. B Biomed. Sci. Appl., Vol. 745, pp. 197-210. Munchbach, M., Quadroni, M., Miotto, G. and James, P. (2000), ‘Quantitation and facilitated de novo sequencing of proteins by isotopic N-terminal labeling of peptides with a fragmentation-directing moiety’, Anal. Chem., Vol. 72, pp. 4047-4057. Mason, D. E. and Liebler, D. C. (2003), ‘Quantitative analysis of modified proteins by LC-MS/MS of peptides labeled with phenyl isocyanate’, J. Proteome Res., Vol. 2, pp. 265-272. Zhang, X., Jin, Q. K., Carr, S. A. and Annan, R. S. (2002), ‘N-terminal peptide labeling strategy for incorporation of isotopic tags: A method for the determination of site-specific absolute phosphorylation stoichiometry’, Rapid Commun. Mass Spectrom., Vol. 16, pp. 2325-2332.), esterification of the C-terminus, incorporation of ¹⁶O/¹⁸O via proteolysis into the C-terminus (Goodlett, D. R., Keller, A., Watts, J. D. et al. (2001), ‘Differential stable isotope labeling of peptides for quantitation and de novo sequence derivation’, Rapid Commun. Mass Spectrom., Vol. 15, pp. 1214-1221. Mirgorodskaya, O. A., Kozmin, Y. P., Titov, M. I. et al. (2000), ‘Quantitation of peptides and proteins by matrix-assisted laser desorption/ionization mass spectrometry using (18)O-labeled internal standards’, Rapid Commun. Mass Spectrom., Vol. 14, pp. 1226-1232. Wang, Y. K., Ma, Z., Quinn, D. F. and Fu, E. W. (2001), ‘Inverse 180 labeling mass spectrometry for the rapid identification of marker/target proteins’, Anal. Chem., Vol. 73, pp. 3742-3750. Stewart, I. I., Thomson, T. and Figeys, D. (2001), ‘¹⁸O labeling: A tool for proteomics’, Rapid Commun. Mass Spectrom., Vol. 15, pp. 2456-2465. Yao, X., Freas, A., Ramirez, J. (2001), ‘Proteolytic ¹⁸O labeling for comparative proteomics: Model studies with two serotypes of adenovirus’, Anal. Chem., Vol. 73, pp. 2836-2842.), and labeling of tryptophan with 2-nitrobenzenesulfenyl chloride, lysine (Cagney, G. and Emili, A. (2002), ‘De novo peptide sequencing and quantitative profiling of complex protein mixtures using mass-coded abundance tagging’, Nat. Biotechnol., Vol. 20, pp. 163-170.) and tyrosine residues (Zhou, H., Watts, J. D., and Aebersold, R. (2001) A systematic approach to the analysis of protein phosphorylation. Nat. Biotechnol. 19, 375-378).

### Example 7

### Loading/Binding of Peptides to Pd-Substrate

The labeled peptides from Example 6 are incubated in a spin tube with a 0.22 μm filter with the conjugated Pd-substrate of Example 1 for 2 hours at 60° C. at pH 3.0. The peptides not bound to the Pd-substrate are collected through the filter by centrifugation. The peptides of the flow-through can be saved and analyzed separately from the peptides bound to the palladium. At this point the Pd-substrate is washed and again the flow-through can be collected. Following washing, the bound peptides are eluted using any of a variety of elution reagents which include but are not limited to guanidine thiocyanate (GuSCN), Na₂S₂O₅, Na₂S₂O₃ and beta-mercaptoethanol. For example, the particles are treated with an equal volume of 4M GuSCN, resulting in a final volume of 2M GuSCN. This solution is incubated at 60° C. for 1 hour. The Pd-substrate is once again separated by centrifugation and the flow-through collected. This flow-through solution containing the targeted peptides can then be desalted by dialysis or reverse phase liquid chromatography.

### Example 8

### Removal of Peptides Containing Methionine/Cysteine, Followed by Loading/Binding of Histidine-containing Peptides to Pd-Substrate

Alternatively, the labeled peptides from Example 6 are incubated in a spin tube with a 0.22 μm filter with a substrate complex that has affinity for methionine and cysteine residues. The peptides not bound to the substrate are collected through the filter by centrifugation. These peptides of the flow-through are saved and analyzed separately (using the method of Example 7) from the peptides bound to the substrate.

### Example 9

### Mass Spectrometry

Both of the Flow-through solutions (targeted and non-targeted peptides) from Examples 7 and 8 are desalted and spotted on MALDI plates with MALDI matrix and subjected to mass spectrometry methods. The samples may also be subjected to LC-MALDI as well as 2D-LC-MS/MS.

What is claimed is:

1. A method for separating proteins or peptides of a sample comprising contacting said sample with a palladium (Pd) coordination compound.

2. The method of claim 1, wherein said Pd coordination compound is stably associated with a substrate.

3. The method of claim 2, wherein said method further comprises recovering said proteins or peptides.

4. The method of claim 3, wherein said proteins or peptides bind to the Pd coordination compound and are recovered by eluting said proteins or peptides.

5. The method of claim 3, wherein said proteins or peptides do not bind to the Pd coordination compound.

6. The method of claim 1, wherein the proteins or peptides are contacted with a cleaving agent, before, after, or while contacting the sample with the Pd coordination compound.

7. The method of claim 6, wherein the cleaving agent comprises trypsin.

8. The method of claim 1, wherein the sample is selected from the group consisting of a cell lysate, plasma, cerebrospinal fluid and urine.

9. The method of claim 1, wherein the sample is contacted with the Pd coordination compound stably associated with the substrate under conditions suitable for binding proteins or peptides comprising sulfur or nitrogen groups.

10. The method of claim 9, wherein the proteins or peptides comprise a methionine residue, a histidine residue, a reduced cysteine residue, a derivatized residue comprising a sulfur or nitrogen group suitable for binding to a coordination site on the Pd compound or a combination thereof.

11. The method of claim 6, wherein the proteins or peptides contacted with the cleaving agent are fragmented to produce peptide ions.

12. The method of claim 3, wherein the mass of a recovered protein or peptide is determined.

13. The method of claim 12, wherein the determined mass is compared to the mass of a known or previously characterized peptide.

14. The method of claim 12, wherein mass is determined using mass spectrometry.

15. The method of claim 3, wherein the amino acid sequence of the recovered protein or peptide is determined.

16. The method of claim 15, wherein the quantity of the protein or peptide is determined.

17. The method of claim 16, wherein the relative quantity of the protein or peptide compared to the quantity of a reference protein or peptide is determined.

18. The method of claim 1, comprising contacting the Pd coordination compound with two differentially labeled samples.

19. The method of claim 18, wherein one sample comprises proteins and/or peptides labeled with a mass-altering label and the other sample is unlabeled.

20. The method of claim 18, wherein one sample comprises proteins and/or peptides labeled with a first mass-altering label and the other sample is labeled with a second mass-altering label.

21. The method of claim 18, wherein the ratio of differentially labeled peptides that are chemically identical except for the presence or absence of the mass altering label is determined.

22. The method of claim 12, wherein a first sample is from a healthy patient while a second sample is from a patient with a disease.

23. The method of claim 1, comprising determining at least one characteristic of the recovered proteins using an analysis system.

24. The method of claim 23, wherein the analysis system comprises a mass spectrometer.

25. A kit comprising a Pd coordination compound and a solid substrate.

26. The kit of claim 25, wherein the Pd coordination compound is stably associated with the solid substrate.

27. The kit of claim 25, wherein said kit further comprises an elution solution for removing a biomolecule bound to the Pd-coordination compound from the Pd-coordination compound.

28. The kit of claim 25, wherein said kit further comprises a cleaving agent.

29. The kit of claim 28, wherein the cleaving agent comprises trypsin.

30. The kit of claim 25, further comprising at least one mass-altering label for labeling a biomolecule.

31. The kit of claim 25, further comprising a pair of mass-altering labels.

32. The kit of claim 31, wherein the pair of mass-altering labels comprises a heavy and light isotope pair.

33. The kit of claim 25, wherein the Pd coordination compound is stably associated with the substrate by a linker covalently bound to the substrate.

34. A system comprising a Pd coordination compound and an analysis system for determining at least one characteristic of a protein or peptide separated from the Pd coordination compound.

35. The system of claim 34, wherein Pd coordination compound is stably associated with a substrate.

36. The system of claim 34, further comprising a separation device for separating proteins or peptides separated from the Pd coordination compound.

37. The system of claim 34, wherein the analysis system comprises a mass spectrometer.

38. The system of claim 34, wherein said Pd-substrate composition is coupled to a column.
